(19) United States
(12) Patent Application Publication
(10) Pub. No.: US 2004/0073214 A1
(43) Pub. Date: Apr. 15, 2004

(54) Spondylolisthesis correction apparatus and method

(76) Inventor: Hamid M. Mehdizadeh, Los Gatos, CA (US)

Correspondence Address: Henry M. Stanley, 165 East Hilton Drive, Boulder Creek, CA, 95006, US

(21) Appl. No.: 10/269,118

(22) Filed: Oct. 10, 2002

(51) Int. Cl. A61B 17/56

(52) U.S. Cl. 606/61

(57) ABSTRACT

Apparatus and process for correcting the condition of spondylolisthesis includes a split correction instrument for moving a displaced vertebra into correct position relative to an adjacent vertebra in conjunction with a disc space spreader that does not impede the corrective movement, but does impede return of the vertebra to its displaced position. The split correction instrument and disc space spreader are used to perform preliminary steps, such as disc space spreading and cutting threads that mate with a stabilizing fusion cage, as well as spondylolisthesis correction and maintenance of the correction prior to stabilization.

### SUMMARY OF THE INVENTION

One aspect of the invention herein relates to apparatus for insertion between a superior vertebra and an inferior vertebra for correcting spondylolisthesis. The structural combination includes an elongate member having an accessible end and an insertion end. The elongate member has a first portion extending from the accessible end to the insertion end and also has a second portion extending from the accessible end to the insertion end. Means is included for engaging the superior and inferior vertebrae on the insertion end. Further, means is included for moving the first portion in lengthwise sliding movement relative to the second portion.

In another aspect of the invention, a disc space spreader is provided for contact with adjacent surfaces on adjacent superior and inferior vertebrae in laminectomy procedures. The disc space spreader is used in cooperation with an insertion/retraction tool. A main spreader body extends along a longitudinal dimension and has a proximal end and a distal end. A wedge extends from the distal end and has opposing converging surfaces terminating at an apex. First and second opposing surfaces are on the main spreader body extending from the proximal end to intercept ones of the opposing converging surfaces. First unidirectional gripping means are formed on the first opposing surface for fixing the main spreader body to the adjacent superior vertebral surface when force normal thereto is applied along the longitudinal dimension in one direction. Second unidirectional gripping means is formed on the second opposing surface for fixing the main spreader body to the adjacent inferior vertebral surface when force normal thereto is applied along the longitudinal dimension in an opposite direction. Further, means is provided for releasably engaging the insertion and retraction tool on the proximal end.

The invention further includes a method of correcting the condition of spondylolisthesis wherein a superior vertebra is displaced relative to an adjacent inferior vertebra in a column of attached vertebrae having a spinal canal extending therethrough. The process includes the steps of inserting a disc space spreader into the intervertebral space between, and to obtain contact with, the displaced superior and inferior vertebrae on one side of the spinal canal. Also included is the step of engaging the displaced superior and inferior vertebrae adjacent surfaces with the disc space spreader to allow relative motion therebetween only in a direction to reduce the displacement. Additionally, the process includes the step of inserting a split surgical tap into the intervertebral space between the displaced superior and inferior vertebrae on an opposite side of the spinal canal, wherein one portion of the split surgical tap is embedded in threads formed on the superior vertebra and another portion of the split surgical tap is embedded in threads formed on the inferior vertebra. The process includes the step of moving the one portion of the split surgical tap in a direction relative to the other portion thereof as permitted by the disc space spreader to reduce the vertebral displacement, together with the step of removing the split surgical tap.

### BRIEF DESCRIPTION OF THE DRAWINGS

FIG. 1 is an elevation showing a human lumbosacral spinal column portion.

FIG. 2 is an elevation showing lumbar spondylolisthesis.

FIG. 3A is a lateral elevation showing a spondylolisthesis condition in the lumbar region.

FIG. 3B is an opposite lateral elevation showing the spondylolisthesis condition of FIG. 3A.

FIG. 3C is a lateral elevation showing spondylolisthesis correction by the present invention.

FIG. 4 is a view along the line 4-4 of FIG. 3A.

FIG. 5 is an elevation of an old art bone tap.

FIG. 6 is a section of the bone tap of the present invention.

FIG. 7 is another section illustrating displacement between the halves of the bone tap of the present invention.

FIG. 8 is a view along the line 8-8 of FIG. 6.

FIG. 9A is an elevation of the disc space spreader of the present invention.

FIG. 9B is a section along the line 9B-9B of FIG. 9A.

FIG. 10 is a section along the line 10-10 of FIG. 6.

### BACKGROUND OF THE INVENTION

The primary bone structure in humans for support of the body trunk is a column of bone segments or vertebrae. Each individual vertebra is connected serially by cartilage that extends between each vertebra and an adjacent one. These connections are referred to as facet joints, existing at the posterior of the vertebrae, one on the right side and one on the left. The facet joints therefore extend between inferior and superior vertebrae. Each vertebra therefore has an inferior and a superior facet portion. Normally a disc is situated between adjacent vertebrae, serving as a shock absorbent member. A channel extends through the column of bone segments through which extends a delicate bundle of nerve paths, the cauda equina, which is enclosed in an envelope or dura.

Bone and cartilage between the inferior and superior facets is referred to as the interarticularis. In four to five percent of observed patients, a condition known as spondylolysis is present. Spondylolysis exists when there is a cut or degeneration (usually in older persons) causing separation in the interarticularis. Spondylolysis is often a precursor to a condition known as spondylolisthesis, to be hereinafter described.

Spondylolisthesis occurs when a superior vertebra shifts forward relative to an inferior vertebra. Reverse spondylolisthesis is also possible but rare. Compression of the cauda equina and nerve roots at the level of the spondylolisthesis is due primarily to pinching of the dural envelope by the displaced vertebra. Thus, when occurring in the lumbar portion of the spine, the clinical picture of symptomatic spondylolisthesis is one of low back pain.

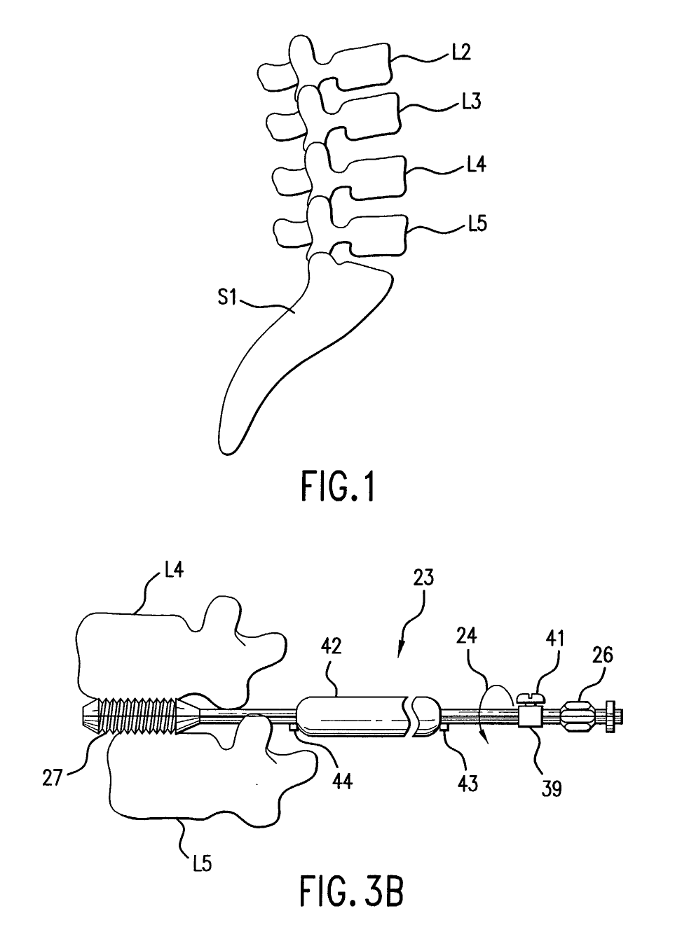

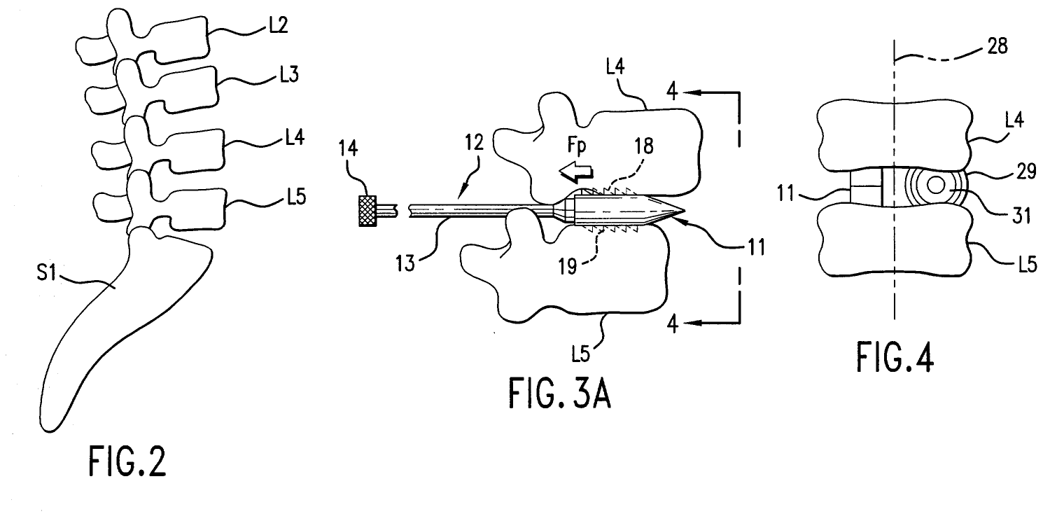

Referring to FIG. 1, an elevation of the lower or lumbosacral portion of the vertebral column is shown. The illustrated vertebrae are in the lumbar region of the human body and are designated in FIG. 1 as L2-L5 from top to bottom. These vertebrae are positioned on top of the sacral region, having an upper member shown as S1 in FIG. 1. Referring now to FIG. 2, it is seen that superior vertebra L4 has shifted forward (to the right in the figure) relative to inferior vertebra L5. This is due to spondylolysis, previously described. Thus, FIG. 2 illustrates the aforementioned condition of spondylolisthesis. There are various grades of spondylolisthesis depending upon how far forward the superior vertebra has shifted relative to the inferior. Currently, the condition is separated into four grades: Grade 1 is a shift of 0-25%, Grade 2 is a shift of 25-50%, Grade 3 is a shift of 50-75%, and Grade 4 illustrates a shift of 75-100% of slippage of the superior vertebra from a position on top or directly above the inferior vertebra.

The vertebral column is stabilized currently through the use of pedicle screws, rod fixations and/or other methods and instrumentation. Prior to stabilization, however, any degree of spondylolisthesis must be corrected. The following describes apparatus and method for accomplishing this aim.

### DESCRIPTION OF THE PREFERRED EMBODIMENTS

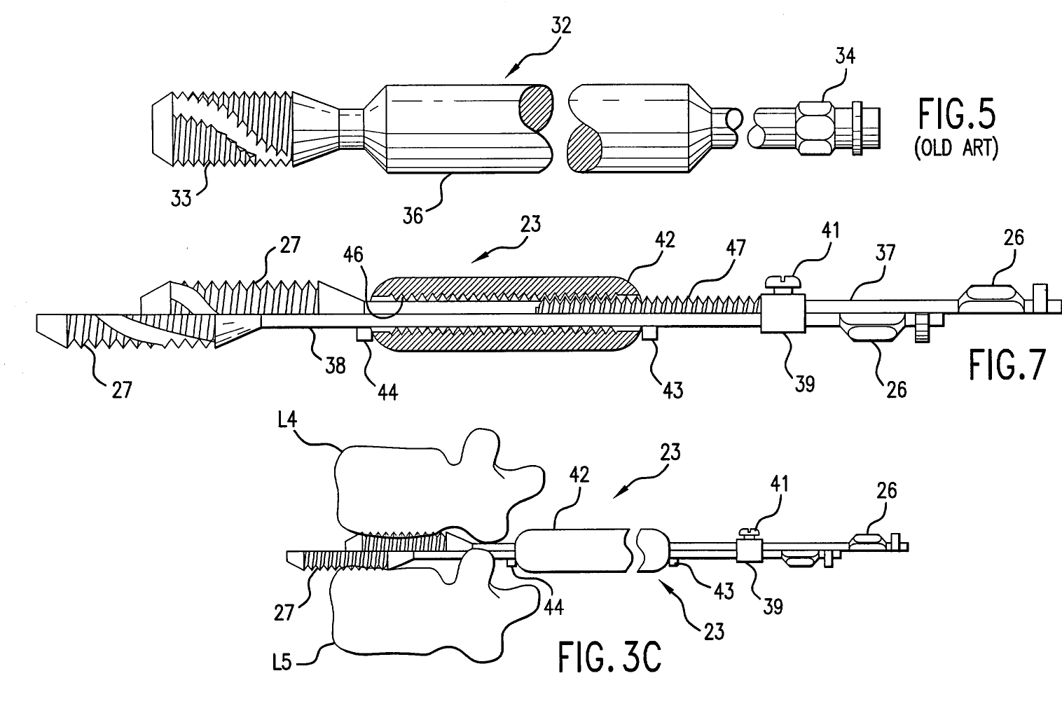

Disc space spreaders are used during laminectomy procedures to maintain a desired space between adjacent vertebrae on one side of the center line of the spine while other laminectomy procedures are in process on the opposing side of the spine center line. A detailed discussion of disc space spreaders is contained in U.S. Pat. No. 6,030,390, owned by the inventor herein, and is incorporated by reference. FIG. 3A shows a disc space spreader 11 engaged by an insertion and retraction tool 12, having a slender shank 13 and a knurled impact cap 14 at one end. As shown in FIGS. 9A and 9B, the slender shank 13 has a threaded end on the end opposite the impact cap 14 that is configured to engage threads in a threaded blind hole 16 in the posterior portion of the disc space spreader 11. A wedge is formed on the anterior end of the disc space spreader 11 by converging upper and lower ramps 17a and 17b, respectively, as seen in FIG. 9A. An upper surface of the disc space spreader has sawtooth shaped teeth 18 formed thereon, with the sharp edges of the sawtooth teeth facing the posterior portion of the disc space spreader. A lower surface of the disc space spreader 11 has an array of sawtooth teeth 19 thereon having sharp edges facing the anterior portion of the disc space spreader as shown. The sawtooth shaped teeth 18 and 19 perform a unidirectional gripping action when engaging surfaces, such as adjacent inferior and superior vertebral surfaces when the disc space spreader is inserted between adjacent vertebrae. A force directed normal to the array of teeth 18 and 19 is one that has a direction parallel to the surface of the disc space spreader on which the arrays of teeth 18 and 19 are formed. Referring again to FIG. 3A, it may be seen that a force Fp directed in a posterior direction will move the vertebra L4 in a posterior direction without resistance from the teeth 18. Similarly, such a normal force exerted on the inferior vertebra L5 in FIG. 3A will be resisted by the sawtooth shaped teeth 19 to keep vertebra L5 fixed in place relative to the disc space spreader 11. The teeth 18 and 19 are shown in phantom lines in FIG. 3A. The insertion/retraction tool 12 is used to insert the disc space spreader between adjacent vertebrae (L4 and L5 in FIG. 3A) by urging the spreader into place with light blows applied to the impact cap 14. Once the spreader is in place, the insertion/retraction tool is disengaged from the threads 16 and removed while laminectomy procedures progress.

If the situation requires, for example in the instance where the space between adjacent vertebrae is very small and must be separated a considerable distance, disc space spreader 11 may be oriented to place smooth sides 21 and 22 (FIG. 9B) in position to contact the inferior surface of vertebra L4 and the superior surface of vertebra L5, respectively, during insertion. As shown in FIG. 9B, the side surfaces and upper and lower surfaces of the disc space spreader are rounded at their intersections. Once the disc space spreader is in place between the vertebrae, the spreader body may be rotated ninety degrees (counterclockwise in FIG. 9B) by manipulation of the insertion/retraction tool 12 so that the sawtooth shaped teeth 18 and 19 are placed in contact with the inferior and superior surfaces of the adjacent vertebrae as shown in FIG. 3A.

With reference now to FIG. 3B, a lateral view of the lumbar vertebrae L4 and L5 is seen taken from the opposite side of the lateral view in FIG. 3A. A bone tap and spondylolisthesis correction instrument 23 configured in accordance with the present invention is shown positioned between the vertebrae L4 and L5 in FIG. 3B, having been inserted between the vertebrae by rotating the instrument 23 in the direction shown by arrow 24. The spondylolisthesis instrument 23 has structure at a proximate end such as the hexagonal formation 26 that may be engaged by a manually operated device to perform the rotation indicated by the arrow 24. At a distal end of the instrument 23 a bone tap 26 is seen thereon having a thread formation to form threads in the inferior surface of vertebra L4 and the superior surface of vertebra L5 as the tap is rotated. The spondylolisthesis correction tool 23 is inserted between vertebrae on the side of the canal running through the spinous process carrying the dura and cauda equina and nerve roots opposite from the side on which the disc space spreader 11 is inserted. The aforementioned channel and its vital content is represented by the center line 28 shown in FIG. 4. FIG. 4 shows the disc space spreader 11 on one side of the center line and a tang on a nerve root retractor 29 on the opposing side. The nerve root retractor 29 and its function is fully described in U.S. Pat. No. 5,803,904, incorporated by reference and invented by the applicant herein. The nerve root retractor 29 is used in various laminectomy procedures including insertion of a threaded fusion cage 31, shown in FIG. 4. The threaded fusion cage 31 is used in some laminectomy procedures and is fully described in U.S. Pat. Nos. 5,928,284 and 6,231,609 both issued to the inventor herein and also incorporated herein by reference. The fusion cage 31 has a variety of sizes and thread configurations to suit the size and requirements of the specific patient as do the threads 27 on the distal end of the spondylolisthesis correction instrument 23. Once the fusion cage 31 is in place and in contact with the threads formed by the correction instrument 23 on the inferior and superior adjacent surfaces of the vertebrae L4 and L5, respectively, the intradiscal space between the adjacent vertebrae is slightly increased and the disc space spreader 11 is now loose between the vertebrae. Consequently, the insertion/retraction tool 12 may be reengaged with the threads in the blind hole 16 (FIG. 9B) and the disc space spreader removed from between the adjacent vertebrae. With the looseness as described, withdrawal of the disc space spreader will not damage the inferior surface of vertebra L4 as the spreader is withdrawn.

FIG. 5 shows an old art bone tap 32 having tapping threads 33 at a distal end and structure 34 at a proximal end for engaging and rotating the bone tap for insertion and thread cutting and also for removal as described previously in conjunction with FIG. 3B. The bone tap of FIG. 5 has a solid member 36 extending between the distal and proximal ends. After the spondylolisthesis is corrected as herein described, the bone tap 32 or the correction instrument of the present invention may be used to form threads on the adjacent surfaces of L4 and L5 that were previously contacted by the disc space spreader 11. Thus, another threaded fusion cage (similar to item 31) can be inserted into the intradiscal space previously occupied by the disc space spreader.

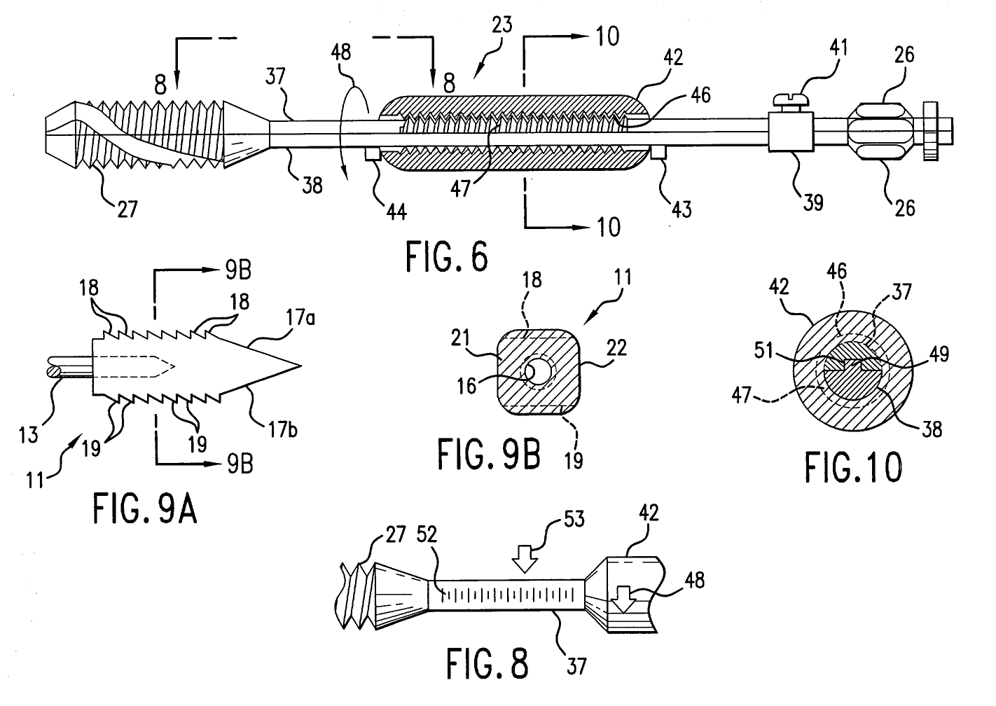

The details of the spondylolisthesis correction instrument 23 construction are seen with reference to FIG. 6 of the drawings. The instrument is separated into an upper portion 37 and a lower portion 38. The separation runs the entire length of the correction instrument from the distal end through the proximal end as seen in FIG. 6. As the instrument is initially put into use, the two portions 37 and 38 are locked together as shown so that there is no discontinuity in the threads 37. The locking is obtained by a locking mechanism located near the proximal end of the instrument consisting of a collar 39 and a compression member or screw 41. In this locked condition the bone tapping threads 37 are inserted between superior and inferior vertebrae as described in conjunction with FIG. 3B.

As also seen in FIG. 6, the spondylolisthesis correction instrument 23 has a centrally located adjustment member 42 that is captured longitudinally on the lower portion 38 of the instrument by a pair of spaced stops 43 and 44 thereon. As a result, when the clamping mechanism represented by the collar 39 and the clamping screw 41 engages the upper and lower portions 37 and 38 of the correction instrument 23, the centrally located member 42 is fixed on the upper 37 portion as well as on the lower portion 38. Central member 42 has a through hole 45 having female threads 46 formed therein. Upper portion 37 of the correction instrument 23 has male threads 47 formed on the length thereof that mate with threads 46. The threads 47 are surrounded by the centrally located member 42 when the instrument is as seen in FIG. 6. It should be noted that the lower portion 38 does not have male threads formed thereon. As a result, when the clamp represented by the collar 39 and the screw 41 is loosened and the centrally located member 42 is rotated in the direction of the arrow 48 as seen in FIG. 6, the upper portion 37 of the correction instrument is moved to the right from the position seen in FIG. 6 toward the position shown in FIG. 7. FIG. 7 represents the instrument with upper and lower portions 37 and 38 displaced to the maximum extent for the illustrated configuration.

The upper and lower portions 37 and 38 of the spondylolisthesis correction instrument 23 are guided in their lengthwise displacement by contacting surfaces such as those displayed in the section of FIG. 10. In this embodiment the upper portion 37 has a groove 49 along the length thereof. The lower portion 38 has a mating land 51 configured to closely fit within the groove 49, wherein the land extends along the length of the lower portion. Adjustment of the centrally located member 42 in the manner described hereinbefore causes the spondylolisthesis correction instrument 23 to approach the configurations shown in FIG. 7 and FIG. 3C. The threads 27 cut threads into the inferior and superior adjacent surfaces of vertebrae L4 and L5 in the part of the process illustrated in FIG. 3B. Subsequent adjustment of the correction instrument 23 by rotation of the centrally located member 42 in accordance with the foregoing description brings the vertebra L4 back into position overlying vertebra L5 as illustrated in FIG. 3C. The cutting threads 27 on the upper and lower portions of the correction instrument therefore act as gripping structure to maintain the inferior vertebra L5 in place while moving the superior vertebra L4 back into correct position with relation to the inferior vertebra. This relative movement may be seen to be unimpeded by the orientation of the sawtooth constructions 18 and 19 on the upper and lower surfaces of the disc space spreader 11 when positioned as viewed in FIG. 3A. Moreover, when the vertebrae L4 and L5 have been corrected in location to that seen in FIG. 3C, the sawtooth formations 18 and 19 prevent the vertebrae L4 and L5 from resuming their previous positions shown in FIGS. 3A and 3B.

FIG. 8 shows a scale 52 etched on the visible surface of the upper portion 37 of the correction instrument 23. The scale 52 may be graduated in increments of the pitch of the bone tap threads 27. In this fashion if the correction is made in whole increments of thread pitch and the correction instrument 23 is then withdrawn by rotation from the condition seen in FIG. 3C, the threads 27 are continuous and during withdrawal will not disrupt the thread pattern previously cut into the respective inferior and superior adjacent surfaces of the vertebrae L4 and L5 during the portion of the process represented by FIG. 3B. An arrow 53 illustrates the position in FIG. 8 where the visible edge of the centrally located member 42 will lie on the scale following completion of the portion of the process illustrated in FIG. 3C. The superior vertebra L4 has there been moved back into correct position to relieve the spondylolisthesis condition relative to L5 by the number of thread pitches for the threads 27 indicated on the scale 52. As mentioned hereinbefore, the thread pitch for the threads 27 vary somewhat, but are generally in the area of 20 mm.

The method of the present invention relates to correction of the condition of spondylolisthesis prior to stabilization of the corrected vertebrae in laminectomy procedures. The process includes insertion of a disc space spreader into the intervertebral space and obtaining contact with the inferior and superior adjacent discs through the use of a disc space spreader as illustrated in FIGS. 9A and 3A. In this fashion the displaced vertebrae are engaged so that relative motion therebetween is allowed only in a direction to reduce the displacement by virtue of the configuration of the sawtooth configurations 18 and 19 on the upper and lower surfaces of the disc space spreader, respectively. The disc space spreader may be inserted on one side of the spinal canal with the smooth sides 21 and 22 (FIG. 9B) of the spreader in contact with the inferior and superior surfaces of the adjacent vertebrae until the spreader reaches the position shown in FIG. 3A if the surgeon believes undue disruption of the contacted surface on the inferior vertebra will occur during insertion. Thereafter the spreader 11 is be rotated through 90 degrees to place the sawtooth configurations 18 and 19 in contact with the adjacent vertebral surfaces as previously described. A split surgical bone tap in the form of the spondylolisthesis correction instrument 23 shown in FIG. 6 is then inserted into the intervertebral space on the opposite side of the spinal canal from the position of the disc space spreader 11. The split surgical bone tap is inserted rotationally into the intervertebral space until one portion of the tap is embedded in threads formed thereby on the superior vertebra and the other portion of the tap is embedded in threads formed on the inferior vertebra (FIG. 3B). One portion 37 of the split surgical tap 23 is then moved in a direction relative to the other portion 38 thereof so as to reduce the vertebral displacement as seen in FIG. 3C. The sawtooth configurations 18 and 19 on the disc space spreader 11 as shown in FIG. 3A do not impede this corrective displacement. The scale 52 on the visible surface of the portion 37 of the correction instrument 23 indicates the correction made in increments of the pitch of the threads 27 on the tap. The surgeon may elect to cause the displacement correction between the adjacent vertebrae to occur in even increments of the pitch of the threads 27. The upper and lower portions of the spondylolisthesis correction instrument 23 are locked together by the collar 39 and the screw 41 and the instrument is then withdrawn from the intravertebral space by engaging the proximal end 26 and rotating the instrument to withdraw the threads from the position between the adjacent vertebrae L4 and L5 seen in FIG. 3C. The configuration of the sawtooth formations 18 and 19 on the surfaces of the disc space spreader that are in contact with the surfaces of the adjacent vertebrae then prevent movement of the vertebrae toward resumption of the condition of spondylolisthesis. It should be noted that in the rare instances where a reverse spondylolisthesis condition exists, the disc space spreader 11 is inverted to place the sawtooth configuration 19 on the upper surface to contact the superior vertebra and the rotation of centrally located member 42 reversed from the direction indicated by arrow 48 (FIG. 6) whereupon the process is performed in the same manner as previously described to correct the condition. It should be noted that when inserting the correction instrument 23 initially into the intervertebral space, the clamping device or screw 41 is set to fix the upper and lower portions 37 and 38 of the instrument together. This condition is illustrated in FIG. 3B. Adjustment of the position of L4 relative to L5 as illustrated in FIG. 3C requires the locking mechanism 41 and 39 to be in the released condition. Upon reaching the desired condition of correction between the adjacent vertebrae and prior to removal of the correction instrument 23 it is appropriate to lock the upper and lower portions 37 and 38 together again. Following withdrawal of the spondylolisthesis correction instrument 23 the single tang nerve root retractor 29 seen in FIG. 4 is inserted with the tang entered into the intradiscal space between the adjacent vertebrae. As described in the previously cited '904 patent, the nerve root retractor protects the cauda equina and the nerve roots and provides visual access to the laminal areas for performing subsequent processes and stabilization of the spine. One such procedure involves insertion of the threaded fusion cage 31, the male threads on the cage mating with the female threads cut by the threads 27 on the spondylolisthesis correction instrument 23. FIG. 4 illustrates this part of the process. Insertion of the threaded fusion cage 31 displaces the adjacent surfaces of the vertebrae sufficiently so that when the insertion/retraction tool 12 engages the threaded hole 16 (FIG. 9B), the disc space spreader 11 is withdrawn from between the vertebrae L4 and L5 without undo disruption of the adjacent vertebral surfaces by the teeth 18 and/or 19.

Although the best mode contemplated for carrying out the present invention has been shown and described herein, it will be understood that modification and variation may be made without departing from what is regarded to be the subject matter of the invention.

What is claimed:

1. Apparatus for insertion between a superior vertebra and an inferior vertebra for correcting spondylolisthesis, comprising
an elongate member having an accessible end and an insertion end,
said elongate member having a first portion extending from said accessible end to said insertion end and a second portion extending from said accessible end to said insertion end,
means for engaging the superior and inferior vertebrae on said insertion end, and
means for moving said first portion in lengthwise sliding movement relative to said second portion.

2. Apparatus as in claim 1, comprising
guide means in contact between said first portion and said second portion for guiding said lengthwise sliding movement.

3. Apparatus as in claim 2, wherein said guide means comprises
a lengthwise groove on said first portion and a lengthwise land on said second portion.

4. Apparatus as in claim 1, wherein said means for engaging comprises
threads formed on said insertion end.

5. Apparatus as in claim 1, wherein said means for moving comprises
a sleeve having an inside surface surrounding a midsection of said first and second portions of said elongate member,
threads formed on said inside surface, and
mating threads on said midsection of said first portion, wherein rotation of said sleeve moves said first portion lengthwise relative to said second portion.

6. Apparatus as in claim 1, comprising
means for releasably locking said first portion to said second portion.

7. Apparatus as in claim 5, comprising
means for retaining said sleeve in lengthwise position on said second portion of said elongate member.

8. Apparatus as in claim 1, further comprising
means for indicating displacement of said first portion relative to said second portion.

9. Apparatus as in claim 4, further comprising
a scale for indicating displacement of said first portion relative to said second portion, said scale having graduations in increments of said means for engaging threads pitch.

10. A disc space spreader for contact with adjacent surfaces on adjacent superior and inferior vertebra in laminectomy procedures in cooperation with an insertion/retraction tool, comprising
a main spreader body extending along a longitudinal dimension and having a proximal end and a distal end,
a wedge extending from said distal end having opposing converging surfaces terminating at an apex,
first and second opposing surfaces on said main spreader body extending from said proximal end to intersect ones of said opposing converging surfaces,
first unidirectional gripping means on said first opposing surface for fixing said main spreader body to the adjacent superior vertebral surface when force normal thereto is applied along said longitudinal dimension in one direction,
second unidirectional gripping means on said second opposing surface for fixing said main spreader body to the adjacent inferior vertebral surface when force normal thereto is applied along said longitudinal dimension in an opposite direction, and
means for releasably engaging the insertion and retraction tool on said proximal end.

11. The disc space spreader of claim 10, wherein said first and second unidirectional gripping means comprise
a first array of sawtooth shaped teeth oriented in one direction, and
a second array of sawtooth shaped teeth oriented in an opposite direction.

12. The disc space spreader of claim 10, comprising
third and fourth opposing surfaces extending between said first and second opposing surfaces, and
rounded surfaces at the intersections of said first and second opposing surfaces with said third and fourth opposing surfaces.

13. A method of correcting the condition of spondylolisthesis, wherein a superior vertebra is displaced relative to an adjacent inferior vertebra in a column of attached vertebrae having a spinal canal extending therethrough, comprising the steps of
inserting a disc space spreader into the intravertebral space between and obtaining contact with the displaced superior and inferior vertebrae on one side of the spinal canal,
engaging the displaced superior and inferior vertebrae adjacent surfaces with the disc space spreader to allow relative motion therebetween only in a direction to reduce the displacement,
inserting a split surgical tap into the intravertebral space between the displaced superior and inferior vertebrae on an opposite side of the spinal canal, wherein one portion of the split surgical tap is embedded in threads formed on the superior vertebra and another portion of the split surgical tap is embedded in threads formed on the inferior vertebra,
moving the one portion of the split surgical tap in a direction relative to the other portion thereof as permitted by the disc space spreader to reduce the vertebral displacement, and
removing the split surgical tap.

14. The method of claim 13, further comprising the steps of
inserting a nerve root retractor in place of the split surgical tap, and
inserting a threaded fusion cage within the intravertebral space in the threads formed by the split surgical tap.

15. The method of claim 13, wherein the disc space spreader has a plurality of sides with engaging teeth on a first pair of opposing sides and smooth surfaces on a second pair of opposing sides, and wherein the step of inserting a disc space spreader between the displaced superior and inferior vertebrae, comprises the step of
inserting the disc space spreader in orientation with the second pair of opposing sides in contact with adjacent vertebral surfaces in the intravertebral space, and
reorienting the disc space spreader thereafter to place the first pair of opposing sides in contact with adjacent vertebral surfaces in the intravertebral space.

16. The method of claim 13, further comprising the step of
measuring the movement of the one portion of the split surgical tap.

17. The method of claim 13, wherein the split surgical tap has a thread pitch and wherein the step of moving the one portion of the split surgical tap comprises the step of
moving the one portion in thread pitch increments.
